(19) United States
(12) Patent Application Publication
(10) Pub. No.: US 2023/0118938 A1
(43) Pub. Date: Apr. 20, 2023

(54) BREATH DETECTING SYSTEM AND BREATH DETECTING MAT THEREOF

(71) Applicant: Yun yun AI Baby camera Co., Ltd., Taipei City (TW)

(72) Inventors: Che-Min LIN, Taipei City (TW); Shih-Yun SHEN, Taipei City (TW); Tzu-Ling LIANG, Taipei City (TW); Meng-Ta CHIANG, Taipei City (TW); Bo-Zong WU, Taipei City (TW); Huan-Yun WU, Taipei City (TW); Hsien-Ching WEI, Taipei City (TW)

(21) Appl. No.: 17/559,505

(22) Filed: Dec. 22, 2021

(30) Foreign Application Priority Data
Oct. 14, 2021 (TW) 110138228

(51) Int. Cl. A61B 5/08; A61B 5/00

(52) U.S. Cl. CPC A61B 5/0816; A61B 5/4806; A61B 5/746; A61B 2503/04

(57) ABSTRACT

A breath detecting system and breath detecting mat thereof are disclosed. The breath detecting mat is placed under bed mattress and has a hollow board, a vibration sensor and a signal processing circuit. The vibration sensor and the signal processing circuit are mounted in the hollow board. The vibration sensor senses the micro-vibrations caused by the breathing of the person is lying on the bed mattress and outputs the breath sensing signal to the signal processing circuit. The signal processing circuit samples the sensing signal according to different moving average points to generate the fast-moving and slow-moving average signals. Since the first fast-moving and slow-moving average signals have many cross points, the signal processing circuit calculates each time difference between every two adjacent cross points. A present breath frequency is calculated according to the time differences. Therefore, the noises of the sensing signal are effectively removed.

### CROSS-REFERENCE TO RELATED APPLICATIONS

This application is based upon and claims priority under 35 U.S.C. 119 from Taiwan Patent Application No. 110138228 filed on Oct. 14, 2021, which is hereby specifically incorporated herein by this reference thereto.

### BACKGROUND OF THE INVENTION

### 1. Field of the Invention

The present invention is related to a breath detecting device, and more particularly to a breath detecting system and breath detecting mat thereof.

### 2. Description of the Prior Arts

The breath detecting devices for the person during sleep are various and complex. The breath detecting devices may roughly be classified to a contactable breath detecting device and a contactless breath detecting device.

Since the contactable breath detecting device has a sensor worn on the person, an available breath sensing signal is easily obtained from the sensor and a breath frequency is accurately calculated accordingly. However, the sensor of the contactable breath detecting device is not suitable for babies or some specific patients such as poor sleep patients. Such a baby or patient may select the contactless breath detecting device to detect his/her breath frequency at sleep. However, the contactless breath detecting device usually obtains a weak breath sensing signal with more noise. Therefore, a complex signal processing circuit and/or a complex algorithm are used in the contactless breath detecting device to accurately calculate the breath frequency, but the cost thereof is relatively increased.

To overcome the shortcomings, the present invention provides a breath detecting system and breath detecting mat thereof to mitigate or to obviate the aforementioned problems.

### SUMMARY OF THE INVENTION

An objective of the present invention is to provide a breath detecting system and breath detecting mat thereof.

To achieve the objective as mentioned above, the host has:

a breath detecting mat having:
a hollow board;
at least one vibration sensor mounted in the hollow board to sense vibrations of the hollow board and output a sensing signal; and
a signal processing circuit mounted in the hollow board and electrically connected to the at least one vibration sensor and a first communication module to obtain the sensing signal, wherein the signal processing circuit has following signal processing steps of:
(a) sampling the sensing signal according to a first moving average point to generate a first fast-moving average signal and sampling the sensing signal according to a second moving average point to generate a first slow-moving average signal, wherein the first moving average point is larger than the second moving average point;
(b) calculating a first time difference between every two adjacent first cross points of the first fast-moving and slow-moving average signals; and
(c) transmitting the time differences through the first communication module; and

a host linking to the first communication module through a second communication module to obtain the first time differences and calculate a present breath frequency according to the first time differences.

With the foregoing description, the breath detecting system of the present invention mainly has the breath detecting mat and the breath detecting mat is placed under the bed mattress. Therefore, the breath detecting mat does not contact the person. When the person is lying down on the bed mattress, the at least one vibration sensor in the hollow board senses the micro-vibrations caused by the breathing of the person and outputs the breath sensing signal to the signal processing circuit. Since the amplitudes of the sensing signal are weak and includes noises therein, the signal processing circuit samples the sensing signal according to different moving average points to generate the first fast-moving and slow-moving average signals. On the time axis, the first fast-moving and slow-moving average signals have many cross points. The signal processing circuit calculates each time difference between the two adjacent cross points and further transmits the time differences to the host. The host calculates the breath frequency according to the time differences. Therefore, the noises of the sensing signal are effectively removed and the breath frequency is calculated accurately.

To achieve the objective as mentioned above, the breath detecting mat has:

a hollow board;

at least one vibration sensor mounted in the hollow board to sense vibrations of the hollow board and output a sensing signal; and

a signal processing circuit mounted in the hollow board and electrically connected to the at least one vibration sensor to obtain the sensing signal, wherein the signal processing circuit has following signal processing steps of:

(a) sampling the sensing signal according to a first moving average point to generate a first fast-moving average signal and sampling the sensing signal according to a second moving average point to generate a first slow-moving average signal, wherein the first moving average point is larger than the second moving average point; and

(b) calculating a first time difference between every two adjacent first cross points of the first fast-moving and slow-moving average signals.

With the foregoing description, the breath detecting mat of the present invention is placed under bed mattress and does not contact the person. When the person is lying down on the bed mattress, the at least one vibration sensor in the hollow board senses the micro-vibrations caused by the breathing of the person and outputs the breath sensing signal to the signal processing circuit. Since the amplitudes of the sensing signal are weak and includes noises therein, the signal processing circuit samples the sensing signal according to different moving average points to generate the first fast-moving and slow-moving average signals. On the time axis, the first fast-moving and slow-moving average signals have many cross points. The signal processing circuit calculates each time difference between the two adjacent cross points. Since a breath frequency can be calculated according to the time differences, the breath detecting mat of the present invention may remove the noises from the sensing signal and calculate the breath frequency accurately.

Other objectives, advantages and novel features of the invention will become more apparent from the following detailed description when taken in conjunction with the accompanying drawings.

### BRIEF DESCRIPTION OF THE DRAWINGS

FIG. 1A is a perspective view of a breath detection mat in accordance with the present invention;

FIG. 1B is a cross-sectional view of FIG. 1A;

FIG. 2 is a schematic and operational view of FIG. 1A;

FIG. 3 is a functional block diagram of the breath detection mat in accordance with the present invention;

FIG. 4A is a waveform graph of a sensing signal of a vibration sensor in accordance with the present invention;

FIG. 4B is a waveform graph showing the fast-moving and slow-moving average signals respectively sampled by the sensing signal of FIG. 4A;

FIG. 4C is a waveform graph of time difference between the fast-moving and slow-moving average signals of FIG. 4B;

FIG. 5A is a waveform graph of another sensing signal of a vibration sensor in accordance with the present invention;

FIG. 5B is a waveform graph showing the fast-moving and slow-moving average signals respectively sampled by the sensing signal of FIG. 5A;

FIG. 5C is a waveform graph of time difference between the fast-moving and slow-moving average signals of FIG. 5B; and

FIG. 6 is a functional block diagram of a host in accordance with the present invention.

### DETAILED DESCRIPTION OF THE PREFERRED EMBODIMENTS

The present invention relates to a breath detecting mat to detect a breath frequency of person at lying status. With multiple embodiments and drawings thereof, the features of the present invention are described in detail as follows.

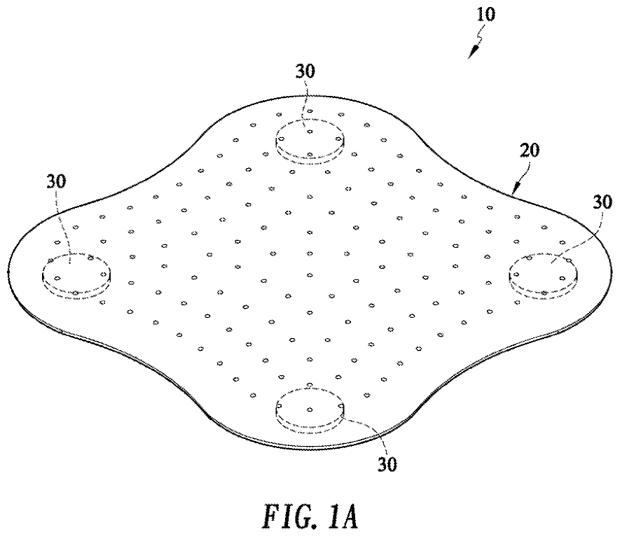

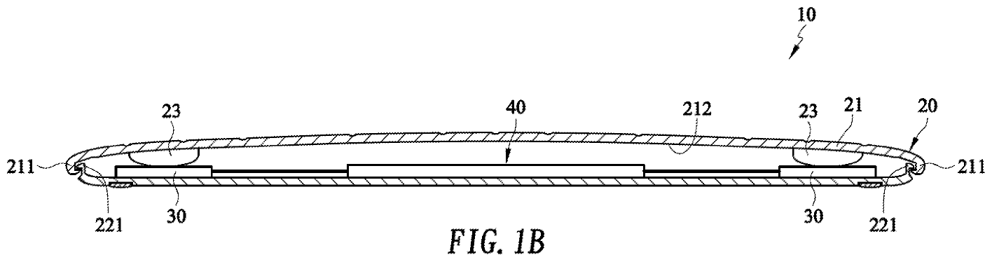

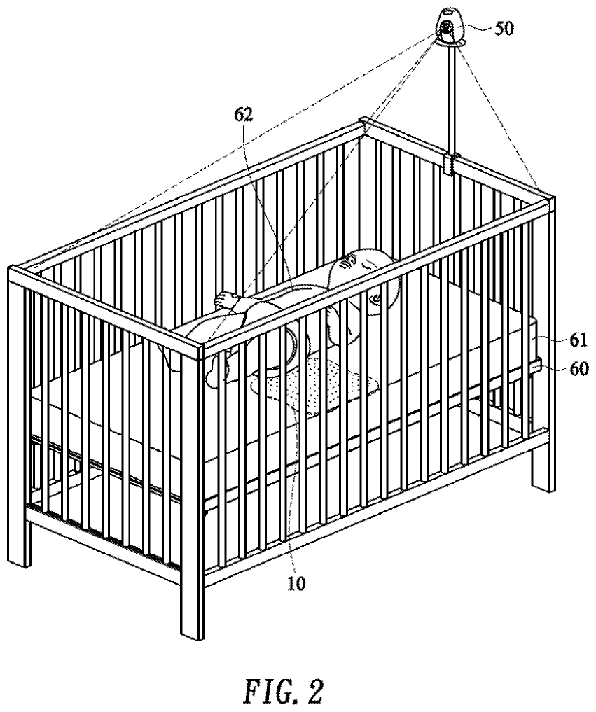

With reference to FIGS. 1A and 2, a breath detecting system has a breath detecting mat 10 and a host 50. The breath detecting mat 10 is used to be placed under a bed mattress on a bed. The breath detecting mat 10 has a hollow board 20, at least one vibration sensor 30 and a signal processing circuit 40. The breath detecting mat 10 may further link to the host.

With further reference to FIG. 1B, the hollow board of the breath detecting mat 10 has a cover 21 and a base 22. The cover 21 matches the base 22 and covers on the base 22. At least one first snap portion 211 is formed on a periphery of the cover 21 and at least one second snap portion 221 corresponding to the first snap portion 211 is formed on a periphery of the base 22. In the present embodiment, the cover 21 and the base 22 are rectangular, and four first snap portions 211 and four second snap portions 221 are respectively formed on the cover 21 and the base 22. Each first snap portion 211 is a down-hook and is integrated and downwardly extended from the periphery of the cover 21. Each second snap portion 221 is an up-hook and integrately and upwardly extends from the periphery of the base 22. The down-hook and the up-hook snaps together and four elastic elements 23 are mounted between the cover 21 and the base 22. Therefore, the cover 21 movably snaps to the base 22. In the present embodiment, the elastic elements 23 are mounted on an inner top of the cover 21.

With further reference FIG. 1B, the least one vibration sensor 30 of the breath detecting mat 10 is mounted on the base 22. In the present embodiment, four vibration sensors 30 are respectively mounted on four corners of the base 22, and respectively correspond and contact to the elastic elements 23 on the cover 21. Each vibration sensor 30 may be a piezoelectric sensor 30′, but not limited to. When the hollow board 20 is placed under the bed mattress 61 on the bed 60 and a person is lying on the bed mattress 61, the cover 21 is periodically and downwardly pressed by the person's breath. That is, the cover 21 moves upward and downward relative to the base 22 and the elastic elements 23 are compressed and released accordingly. Therefore, the vibration sensor 30 contacted with the elastic element 23 senses the person's breath and outputs a sensing signal accordingly.

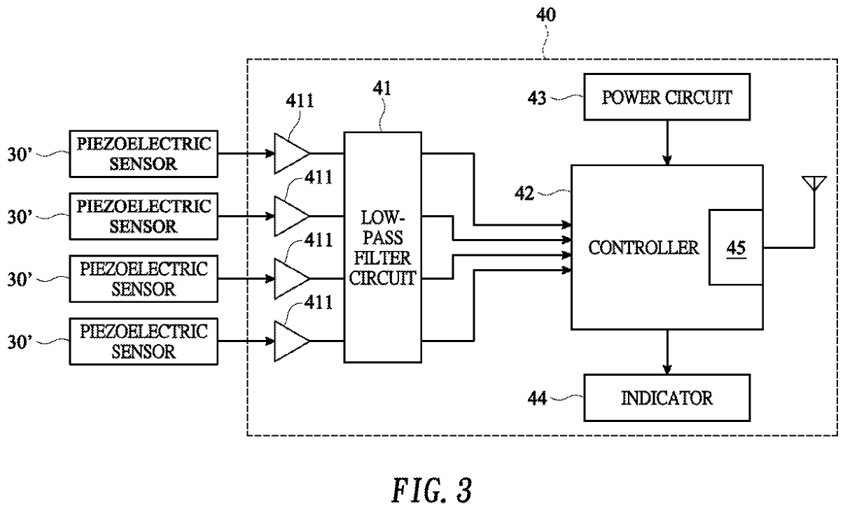

With further reference to FIG. 1B, the signal processing circuit 40 of the breath detecting mat 10 is mounted on the base 22 and electrically connected to each vibration sensor 30 to receive the sensing signal. In the present embodiment, as shown in FIG. 3, the signal processing circuit 40 has a low-pass filter 41, a controller 42, a power circuit 43, an indicator 44 and a first communication module 45. The controller 42 is electrically connected to each piezoelectric sensor 30′ through the low-pass filter 41. The power circuit 43 supplies an operation power to the piezoelectric sensors 30′, the low-pass filter 41, the controller 42, the indicator 44 and the first communication module 45. The indicator 44 is electrically connected to the controller 42 and controlled to display a power status by the controller 42. The first communication module 45 is electrically connected to the controller 42 and dual-links to the host 50. In the present embodiment, the indicator 44 may be an LED indicator and display different light colors to show different power statuses. The first communication module 45 may be a wireless communication module and built in the controller 42. The wireless communication module is a Bluetooth module, but not limited to. The controller 42 has a signal processing procedure having following steps (a) to (c).

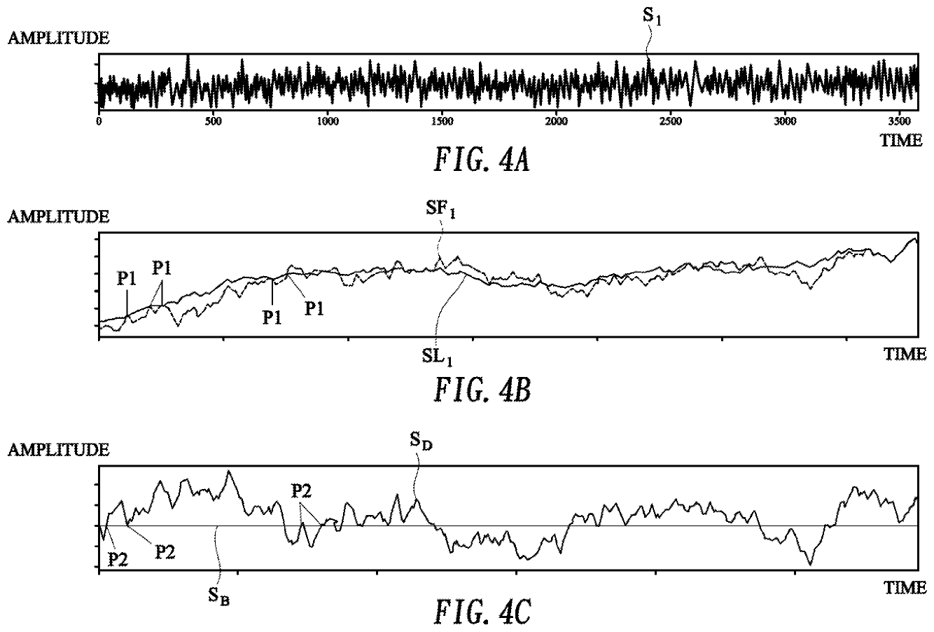

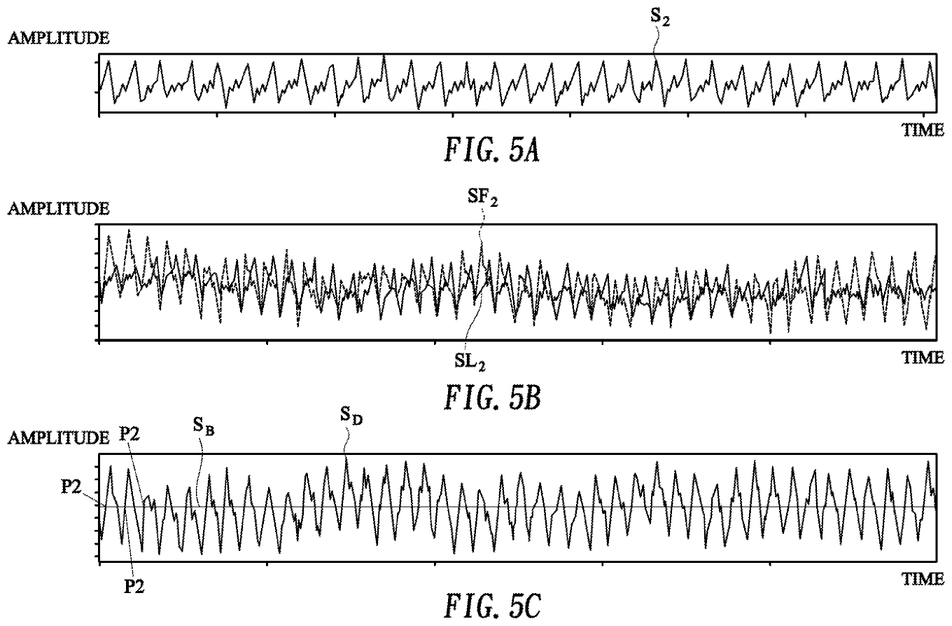

In the step (a), when the controller 42 receives the sensing signal S1 or S2 as shown in FIGS. 4A and 5A, the controller 42 respectively samples the sensing signal S1 or S2 to generate a first fast-moving average signal SF₁ or SF₂ and a first slow-moving average signal SL₁ or SL₂ according to a first moving average point and a second moving average point as shown in FIGS. 4B and 5B. The first moving average point is larger than the second moving average point. In one embodiment, when the breath detecting mat 10 has four piezoelectric sensors 30′ and controller 42 receives four sensing signals, the controller 42 may only select one sensing signal with a maximum amplitude to sample. In detail, during each time interval (T), the maximum values S(i) of the standard deviations of the four sensing signals are compared to determine a k-th sensing signal with a largest maximum values S(k), so the k-th sensing signal is selected to sample.

In the step (b), the controller 42 determines a plurality of first cross points P1 between the first fast-moving average SF₁ or SF₂ and the first slow-moving average signals SL₁ or SL₂. After then, the controller 42 calculates a first time difference between every two adjacent first cross points P1. The first time differences are used to calculate a present breath frequency. In the present embodiment, the controller 42 may generate a difference signal S_{D} by subtracting the first slow-moving average signal SL₁ or SL₂ from the first fast-moving average signal SF₁ or SF₂. The controller 42 presets a reference signal S_{B} and determines a plurality of second cross points P2 between the difference signal S_{D} and the reference signal S_{B}. The controller 42 calculates a second time difference between every two adjacent second cross points P2 and the second time difference is used as the first time difference. The controller 42 may further determine whether a slope of the difference signal S_{D} corresponding to each second cross point relative to the reference signal S_{B} is positive or negative. The controller 42 selects the second cross points P2 corresponding to the positive slope and calculates a third time difference between every two adjacent second cross points P2 corresponding to the positive slope. The third time difference is used as the first time difference.

In the step (c), the controller 42 transmits the first time differences of the step (b) to the host 50 and the host 50 calculates the present breath frequency. In the present embodiment, the controller 42 transmits the third time differences of the step (b) to the host 50. In one embodiment, the controller 42 may also directly calculate the present breath frequency by calculating the first or third time differences. In another embodiment, the controller 42 may further transmit the present breath frequency to the host 50.

With further reference to FIG. 4A, the sensing signal S1 is generated when no person 62 is lying on the bed mattress 61 and the sensing signal S1 is received by the controller 42. The first fast-moving and slow-moving average signals SF₁, SL₁ are generated by the controller 42 according to the first and second moving average points. As shown in FIG. 4B, the first cross points P1 between the first fast-moving and slow-moving average signals SF₁, SL₁ are not periodically distributed on the time axis. Therefore, when the controller 42 subtracts the first slow-moving average signal SL₁ from the first fast-moving average signal SF₁ to generate the difference signal S_{D} and compares the difference signal S_{D} and the reference signal S_{B}, the second cross points P2 between the difference signal S_{D} and the reference signal S_{B} are also not periodically distributed on the time axis, as shown in FIG. 4C.

With reference to FIG. 5, the sensing signal S2 is generated when a person 62 is lying on the bed mattress 61 and the sensing signal S2 is received by the controller 42. The first fast-moving and slow-moving average signals SF₂, SL₂ are generated by the controller 42 according to the first and second moving average points. As shown in FIG. 5B, the first cross points P1 between the first fast-moving and slow-moving average signals SF₂, SL₂ are periodically distributed on the time axis. Therefore, when the controller 42 subtracts the first slow-moving average signal SL₂ from the first fast-moving average signal SF₂ to generate the difference signal S_{D} and compares the difference signal S_{D} and the reference signal S_{B}, the second cross points P2 between the difference signal S_{D} and the reference signal S_{B} are also periodically distributed on the time axis, as shown in FIG. 5C. Therefore, the controller 42 calculates the present breath frequency of the person according to the second cross points P2.

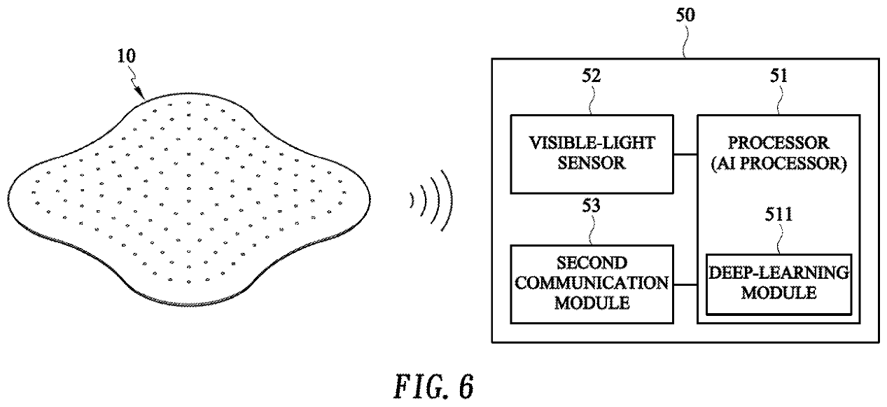

With reference to FIG. 6, the host 50 has a processor 51 and a second communication module 53. The processor 51 is electrically connected to the second communication module 53. When the processor 51 dual-links to the first communication module 45 of the breath detecting mat 10 through the second communication module 53, the processor 51 may receive the first time differences, the second time differences or the third time differences calculated by the breath detecting mat 10. Therefore, the host 50 may calculate the present breath frequency according to the adjacent time differences. In addition, the processor 51 may further determine whether the present breath frequency is normal or abnormal. For baby, the host presets a normal breath frequency range is between 40 and 60 times per minute. That is, if the processor 51 determines the present breath frequency is in the normal breath frequency range, the present breath frequency is determined to be normal. On the contrary, if the processor 51 determines the present breath frequency is out of the normal breath frequency range, the present breath frequency is determined to be abnormal.

In another embodiment, the host 50 may use an AI processor 51′ as the processor 51 and further has a visible-light sensor 52. The AI processor 51′ is electrically connected to the visible-light sensor 52 and the second communication module 53. A deep-learning module 511 is built in the AI processor 51′ and the deep-learning module 511 learns about person, such as eyes, mouth, hands, feet etc. to identify the person's body features. The AI processor 51′ dual-links to the first communication module 45 of the breath detecting mat 10 through the second communication module 53. When the AI processor 51′ determines that the present breath frequency is abnormal, the AI processor further receive a present bed mattress image from the visible-light sensor. The AI processor 51′ determines whether any one of the person's body features exists in the present bed mattress image by the deep-learning module 511. If no person's body feature exists, the AI processor 51′ determines that no person is lying on the bed mattress and does not output an alarm signal. On the contrary, if the person's body feature is identified, the processor 51′ determines that the person's breath is weak or has no breath and outputs the alarm signal immediately.

Since some of breath frequencies at a moving status of the person's body may be calculated by the AI processor 51′, the AI processor 51′ may have a misjudgment of the final present breath frequency. To reduce the misjudgment, the AI processor 51′ may further preset a third moving average point to sample the breath frequencies and generate a moving average value of the breath frequencies. When the AI processor 51′ determines the final present breath frequency according to the moving average value of the breath frequencies, the accuracy of the present breath frequency is increased accordingly.

Based on the foregoing description, the breath detecting system of the present invention mainly has the breath detecting mat, and the breath detecting mat is placed under bed mattress. Therefore, the person is lying on the bed mattress does not directly contact the breath detecting mat. The at least one vibration sensor in the hollow board senses the micro-vibrations caused by the breathing of the person and outputs the breath sensing signal to the signal processing circuit. Since the amplitudes of the sensing signal are weak and includes noises therein, the signal processing circuit samples the sensing signal according to different moving average points to generate the first fast-moving and slow-moving average signals. On the time axis, the first fast-moving and slow-moving average signals have many cross points. The signal processing circuit calculates each time difference between the two adjacent cross points and further transmits the time differences to the host. The host calculates the breath frequency according to the time differences. Therefore, the noises of the sensing signal are effectively removed and the breath frequency is calculated accurately.

Even though numerous characteristics and advantages of the present invention have been set forth in the foregoing description, together with the details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

1. A breath detecting system, comprising:
a breath detecting mat having:
a hollow board;
at least one vibration sensor mounted in the hollow board to sense vibrations of the hollow board and output a sensing signal; and
a signal processing circuit mounted in the hollow board and electrically connected to the at least one vibration sensor and a first communication module to obtain the sensing signal, wherein the signal processing circuit has following signal processing steps of:
(a) sampling the sensing signal according to a first moving average point to generate a first fast-moving average signal and sampling the sensing signal according to a second moving average point to generate a first slow-moving average signal, wherein the first moving average point is larger than the second moving average point;
(b) calculating a first time difference between every two adjacent first cross points of the first fast-moving and slow-moving average signals; and
(c) transmitting the time differences through the first communication module; and
a host linking to the first communication module through a second communication module to obtain the first time differences and calculate a present breath frequency according to the first time differences.

2. The breath detecting system as claimed in claim 1, wherein the step (b) further comprises following acts of:
(b1) generating a difference signal by subtracting the first slow-moving average signal from the first fast-moving average signal; and
(b2) determining a plurality of second cross points between the difference signal and a reference signal, wherein a second time difference between every two adjacent second cross points is used as the first time difference of the step (b).

3. The breath detecting system as claimed in claim 2, wherein in the act (b2), a slope of the difference signal corresponding to each second cross point relative to the reference signal is further determined to be positive or negative, wherein if the positive slope is determined, the second cross points corresponding to the positive slope are selected and a third time difference between every two adjacent selected second cross points is calculated to be used as the first time difference of the step (b).

4. The breath detecting system as claimed in claim 1, wherein in the step (a), when the signal processing circuit receives a plurality of sensing signals, a standard deviation of each sensing signal is calculated, wherein during each time interval, one of the sensing signals with a largest maximum value is selected to be sampled.

5. The breath detecting system as claimed in claim 2, wherein the hollow board comprises a cover and a base on which the cover covered; wherein
the cover has a first periphery and a first snap portion integrately and downwardly extended from the first periphery; and
the base has a second periphery and a second snap portion corresponding to the first snap portion integrately and upwardly extended from the second periphery.

6. The breath detecting system as claimed in claim 5, wherein the signal processing circuit further comprises:
a low-pass filter circuit electrically connected to the at least one vibration sensor to filter low-frequency noise of the sensing signal from the at least one vibration sensor;
a controller electrically connected to the first communication module and the low-pass filter circuit to receive the filtered sensing signal and executing the signal processing steps; and
a power circuit electrically connected to the least one vibration sensor, the low-pass filter circuit and the controller.

7. The breath detecting system as claimed in claim 6, wherein the signal processing circuit further comprises an indicator electrically connected to the controller.

8. The breath detecting system as claimed in claim 6, wherein the host comprises:
a processor; and
a second communication module electrically connected to the processor to dual-link to the first communication module.

9. The breath detecting system as claimed in claim 8, wherein
the processor is an AI processor having a deep-learning module, wherein the deep-learning module identifies whether a person's body feature exits in a photo image; and
a visible-light sensor is electrically connected to the AI processor and outputs the photo image to the AI processor; wherein
the AI processor further determines whether the present breath frequency is in or out of a normal breath frequency range, wherein if the present breath frequency is out of the normal breath frequency range, the AI processor receives the photo image from the visible-light sensor and determines whether the person's body feature exits in the photo image, wherein if a determining result is positive, the AI processor outputs an alarm signal.

10. The breath detecting system as claimed in claim 9, wherein after obtaining the first time differences from the breath detecting mat, the AI processor samples the first time differences according to a third moving average point and generates a moving average value of the first time differences, wherein the AI processor calculates the present breath frequency according to the moving average value of the first time differences.

11. A breath detecting mat, comprising:
a hollow board;
at least one vibration sensor mounted in the hollow board to sense vibrations of the hollow board and output a sensing signal; and
a signal processing circuit mounted in the hollow board and electrically connected to the at least one vibration sensor to obtain the sensing signal, wherein the signal processing circuit has following signal processing steps of:
(a) sampling the sensing signal according to a first moving average point to generate a first fast-moving average signal and sampling the sensing signal according to a second moving average point to generate a first slow-moving average signal, wherein the first moving average point is larger than the second moving average point; and
(b) calculating a first time difference between every two adjacent first cross points of the first fast-moving and slow-moving average signals.

12. The breath detecting mat as claimed in claim 11, wherein the signal processing circuit further has a step of (c) calculating a present breath frequency according to the first time differences of the step (b).

13. The breath detecting mat as claimed in claim 12, wherein
the hollow board further has a communication module electrically connected to the signal processing circuit; and
the signal processing circuit further has a step of (d) transmitting the present breath frequency of the step (c) through the communication module.

14. The breath detecting mat as claimed in claim 11, wherein the step (b) further comprises following acts of:
(b1) generating a difference signal by subtracting the first slow-moving average signal from the first fast-moving average signal; and
(b2) determining a plurality of second cross points between the difference signal and a reference signal, wherein a second time difference between every two adjacent second cross points is used as the first time difference of the step (b).

15. The breath detecting mat as claimed in claim 14, wherein in the act (b2), a slope of the difference signal corresponding to each second cross point relative to the reference signal is further determined to be positive or negative, wherein if the positive slope is determined, the second cross points corresponding to the positive slope are selected and a third time difference between every two adjacent selected second cross points is calculated to be used as the first time difference of the step (b).

16. The breath detecting mat as claimed in claim 11, wherein in the step (a), when the signal processing circuit receives a plurality of sensing signals, a standard deviation of each sensing signal is calculated, wherein during each time interval, one of the sensing signals with a largest maximum value is selected to be sampled.

17. The breath detecting mat as claimed in claim 11, wherein the hollow board comprises a cover and a base on which the cover covered; wherein
the cover has a first periphery and a first snap portion integrately and downwardly extended from the first periphery; and
the base has a second periphery and a second snap portion corresponding to the first snap portion integrately and upwardly extended from the second periphery.

18. The breath detecting mat as claimed in claim 17, wherein the signal processing circuit further comprises:
a low-pass filter circuit electrically connected to the at least one vibration sensor to filter low-frequency noise of the sensing signal from the at least one vibration sensor;
a controller electrically connected to the first communication module and the low-pass filter circuit to receive the filtered sensing signal and executing the signal processing steps; and
a power circuit electrically connected to the least one vibration sensor, the low-pass filter circuit and the controller.

19. The breath detecting mat as claimed in claim 18, wherein the signal processing circuit further comprises an indicator electrically connected to the controller.
